(19) United States
(12) Patent Application Publication
(10) Pub. No.: US 2005/0070463 A1
(43) Pub. Date: Mar. 31, 2005

(54) Use of gram-negative bacterial membrane fraction for inducing the maturation of dendritic cells

(76) Inventors: Christine Libon, Saint Julien En Genevois (FR); Pascale Jeannin, Genevois (FR); Jean-Yves Bonnefoy, Le Sappey (FR)

Correspondence Address: HELLER EHRMAN WHITE & MCAULIFFE LLP, 1666 K STREET,NW, SUITE 300, WASHINGTON, DC, 20006, US

(21) Appl. No.: 10/471,733

(22) PCT Filed: Mar. 14, 2002
(86) PCT No.: PCT/FR02/00906

(30) Foreign Application Priority Data
Mar. 15, 2001 (FR) 01/3518

(51) Int. Cl. A61K 38/16; A61K 31/739

(52) U.S. Cl. 514/8; 514/54

(57) ABSTRACT

The invention relates to the use of a gram-negative bacterial membrane fraction, especially Klebsiella pneumoniae, for inducing the maturation of dendritic cells.

The present invention relates to the use of a membrane fraction of Gram-negative bacteria, in particular Klebsiella pneumoniae, for inducing the maturation of dendritic cells and thus promoting the development of a specific immune response.

Dendritic cells play a role in the development of an immune response and in the initiation of a specific T lymphocyte response (Steinman R M et al Immuno Rev (1997) 156, 25 & Cella M et al Curr Opin Immunol (1997) 9, 10). Immature dendritic cells are located in nonlymphoid tissues. At the level of the skin, in all the epidermes apart from the intestine, they are then called Langerhans' cells; dendritic cells, in a smaller quantity, are also located in organs such as the lung, the liver and the intestine. These immature dendritic cells capture and digest antigens in a highly efficient manner. After an antigenic stimulation in vivo or a stimulation by proinflammatory molecules, the dendritic cells which have captured the antigens migrate into the secondary lymphoid organs. During this migration, the dendritic cells undergo functional and phenotypic modifications which are grouped under the term maturation. This maturation is characterized by an increase in the surface of the dendritic cells of molecules involved in the activation of T lymphocytes (such as CD40, CD54, CD58, CD86 and MHC class I/II), the production of proinflammatory cytokines (IL-6) and of chemokines (such as TNFα, IL-8, MCP-1 and MIP-1a) and lose their capacity to process the antigen. Chemokines recruit inflammatory cells and promote the initiation of an immune response. In particular, IL-8, MCP-1 and MIP-1α attract not only inflammatory cells but also naive and memory T lymphocytes. Thus, dendritic cells, by secreting chemokines, increase their chances of coming into contact with T lymphocytes. The proinflammatory cytokines will also act by directly activating the DCs.

These mature dendritic cells present the antigens to the T lymphocytes and initiate the specific T responses in a very efficient manner, the costimulation molecules being expressed in a large quantity on these cells. The cells, on becoming mature, lose their capacity to capture and process the antigen. In the thymol-dependent zones of the lymphoid organs, dendritic cells which have migrated have acquired powerful immunostimulatory properties and will therefore very efficiently activate the circulating naive T lymphocytes.

The membrane fraction of K. pneumoniae I145 enters into the composition of a pharmaceutical preparation preventing the onset and the recurrence of respiratory infections of bacterial origin and used in humans for 20 years. In this regard, there is a period of nontoxicity of the product.

In application FR9903154, it has been shown that FMKp or one of its major constituents, the outer membrane protein OmpA, called P40 was capable of inducing the production in particular by monocytes of TNF-α and of IL-12, cytokines involved in the antitumor immune response.

In application FR9903153, it has been shown that said membrane fraction FMKp combined with an antigen not only had the capacity to reorient the cytokine response to a Th1/Th2 profile, this being regardless of the mode of administration of said membrane fractions.

The authors of the present invention have now demonstrated that a membrane fraction of Gram-negative bacteria, preferably of Klebsiella pneumoniae, induces the maturation of human dendritic cells, thus allowing the development of a specific T lymphocyte response.

The present invention thus relates to the use of at least one membrane fraction of Gram-negative bacteria, preferably of Klebsiella pneumoniae, for inducing the maturation of dendritic cells.

The present invention also relates to the use of at least one membrane fraction of Gram-negative bacteria, in particular of Klebsiella pneumoniae, for the manufacture of a medicament intended for inducing the maturation of dendritic cells.

The expression membrane fraction of a bacterium is understood to mean in the present invention any purified or partially purified membrane fraction or extract obtained from a culture of said bacterium and whose method of preparation comprises at least one step for lysis of the bacteria obtained after culture and a step for separating the fraction containing the membranes of said bacteria from the total lysate obtained after the lysis step, in particular by centrifugation or filtration. Thus, for the purposes of the present invention, a membrane fraction comprises at least membrane proteoglycans. Thus, for the purposes of the present invention, a membrane fraction may also be defined as a fraction comprising at least membrane fractions combined with LPSs (lipopolysaccharides), known to a person skilled in the art.

The proteoglycan titer of the membrane fractions according to the invention is represented by the sum of the contents of galactose and of proteins, is preferably between:

for galactose: between 1.2 g/l and 3.4 g/l;

for proteins: between 7.5 g/l and 14.9 g/l.

More preferably, this titer will be:

for galactose: between 1.8 g/l and 2.6 g/l;

for proteins: between 9.3 g/l and 11.7 g/l.

The membrane fractions according to the invention can be obtained by the methods described below or by any other equivalent method, in particular those described in patent applications FR9903154 and FR9903153.

These membrane fractions may be used at concentrations of from 0.1 to 100, preferably from 1 to 50, and more preferably from 1 to 20 micrograms per milliliter of culture medium.

In a preferred embodiment of the invention, the Gram-negative bacteria are chosen from bacteria of the genus Klebsiella and preferably of the species pneumoniae.

In a preferred embodiment of the invention, the membrane fractions according to the invention can be used to promote the maturation of dendritic cells in vitro, it being possible for the mature cells to then be reinjected in vivo.

In a still more preferred embodiment of the invention, this medicament can be used to promote the generation and the maturation of dendritic cells in vitro, after bringing into contact with a biological agent. Thus, the invention relates to the use of a membrane fraction according to the invention and additionally of at least one biological agent for the manufacture of a medicament. The latter can for example increase the immunological response toward this biological agent.

This biological agent may be chosen from nucleic acids, proteins, lipids, lipopeptides or polysaccharides.

It may be more particularly chosen from vaccine antigens and/or the antigens of bacteria, viruses, yeasts, parasites and fungi.

More preferably, the biological agent is a tumor antigen. For the purposes of the present invention, a tumor antigen is defined as a tumor protein or peptide, in particular as an epitope, in particular a CTL epitope (peptide sequences interacting with the class I molecules and presented to the CD8+T lymphocytes) or as the nucleic sequence encoding such proteins, peptides or epitopes. The following tumor antigens may be mentioned without limitation: MAGE-2, MAGE-3, MUC-1, MUC-2, HER-2, GD2, carcinoembryonic antigen (CEA), TAG-72, ovarian-associated antigens OV-TL3 and MOV18, TUAN, alpha-feto protein (AFP), OFP, CA-125, CA-50, CA-19-9, renal tumor-associated antigen G250, EGP-40 (or EPCAM), S100 (malignant melanoma-associated antigen), p53, prostate tumor-associated antigens (e.g., PSA and PSMA), and p21ras.

The biological agent may also be chosen from a lysate of autologous and/or heterologous tumor cells. For the purposes of the present invention, the expression autologous tumor cells is understood to mean tumor cells belonging to the subject which is going to receive the compositions according to the invention. The expression heterologous tumor cells should be understood to mean cells derived from tumors obtained from an individual different from the one for whom the composition according to the invention is intended. The use of heterologous cells makes it possible to obtain pharmaceutical compositions which make it possible in particular to treat patients suffering from cancer from whom the collection of tumor cells is not possible. The use of heterologous cells also makes it possible to obtain standard compositions according to the invention comprising antigens found in numerous types of cancer and which can thus be used in a majority of patients.

The tumor cells may be obtained following a removal of cancerous tissues, for example following a biopsy or surgical resection. These cells can then be used as they are or can be cultured before being lysed. A cell lysate can be defined for the purposes of the present invention as a mixture of intracellular and/or membrane, preferably intra and membrane, antigens. Said lysate of autologous and/or heterologous tumor cells according to the invention may be obtained by mechanical, chemical or enzymatic lysis of tumor cells. To lyse the cells mechanically, there may be mentioned in particular the techniques known to a person skilled in the art, namely in particular sonication, ultrasonication or freeze/thaw. Freeze/thaw is most particularly preferred, and most particularly the use of several freeze/thaw cycles. It is also possible to lyse the cells using chemical compounds or enzymes, such as for example a lysis buffer containing digitonin, triton X-100 or Nonidet P40. Any method which makes it possible to break the cell membrane of tumor cells can be used in order to obtain a lysate.

The dendritic cells are thus modified using these cellular lysates or antigens so that they express tumor antigens. Thus, said medicament can be injected at the same time as the dendritic cells or preferably said medicament can be used in vitro, in order to promote the maturation of dendritic cells before reinjecting them into the patients.

The administration of a membrane fraction of Gram-negative bacteria at the same time as the antigen will make it possible to increase the presentation of the antigen by the dendritic cells and thereby the efficacy of the therapeutic treatment.

Thus, by virtue of their efficacy in presenting the antigens and in stimulating the immune system, the dendritic cells in presence of a membrane fraction of Gram-negative bacteria can be used to generate anticancer CTL responses (Nestle F. O. et al., 1998, Nat. Med., 4, 328-332). This approach, called “ex vivo”, therefore consists in loading the dendritic cells ex vivo with the antigen of interest (peptides or cell lysate) in the presence of a membrane fraction of Gram-negative bacteria and in reimplanting these cells in the patient. A step for washing the dendritic cells so as to remove the membrane fraction of Gram-negative bacteria from the medium containing the dendritic cells may be carried out before reimplanting these cells in the patient.

Another approach according to the invention consists in transfecting ex vivo the dendritic cells with the gene encoding the antigen of interest and in particular with a gene encoding an antigen of a bacterium, virus, yeast, parasite, fungus, or a tumor antigen, such as those described above, and/or with a gene encoding a cytokine or a growth factor, such as those described below, and in placing the dendritic cells, before and/or during and/or after the transfection, in the presence of a membrane fraction of Gram-negative bacteria and in reinjecting these transfected cells (Gilboa E. et al., 1998, Cancer Immunol. Immunother., 46, 82-87). A step of washing the dendritic cells so as to remove the membrane fraction of Gram-negative bacteria from the medium containing the dendritic calls may be carried out before reimplanting these cells in the patient. Such approaches have been successfully used in mice and in humans (Hsu F. J. et al., 1996, Nat. Med., 2, 52-58).

The medicament according to the invention may additionally comprise a cytokine or a growth factor, in particular alpha- or gamma-interferon, TNFα, GM-CSF, IL-2, IL-12, IL-4, IL-6 and IL-18, an HSP (Heat Shock Protein) such as for example hsp70, hsp90, hsp96, which makes it possible to potentiate the immune response; and/or fibroblasts genetically modified so as to release a cytokine or a growth factor. There may be mentioned GM-CSF expressing fibroblasts marketed by the company Immune Response Corporation. The use of a membrane fraction of Gram-negative bacteria with at least one biological agent combined with TNF alpha may be used for the manufacture of a medicament intended for increasing the proliferation of T lymphocytes.

The medicament according to the invention may additionally comprise an adjuvant which makes it possible to increase the immune response, in particular chosen from the group of adjuvants comprising MPL-A, Quil-A, ISCOM, CpG, Leif, CT (cholera toxin) or LT (LT for “Heat labile enterotoxin”), as well as the detoxified versions of CT or LT, or bacterial membrane proteins such as the OMPCs of Neisseria meningitidis (Vella et al., Infect. Immun. 60, 1992, 4977-4983), TraT of Escherichia coli (Croft et al., J. Immunol. 146, 1991, 793-798) or PorB of Neisseria meningitidis (Fusco et al., J. Infect. Dis. 175, 1997, 364-372), and preferably an OmpA of a bacterium of the genus Klebsiella, a major outer membrane protein called P40, having an adjuvant activity for peptide subunit antigens (WO 95/27787 and WO 96/14415; Haeuw et al., Eur. J. Biochem. 255, 1998, 446-454; Plotnicky-Gilquin et al., J. Virol. 73, 1999, 5637-5645).

The medicament according to the invention may also comprise a pharmaceutically acceptable medium. For the purposes of the present invention, the pharmaceutically acceptable medium is the medium in which the compounds of the invention are administered, preferably a medium for injection into humans. It may consist of water, an aqueous saline solution or an aqueous solution based on dextrose and/or glycerol.

The medicament according to the invention may additionally be carried in a form which makes it possible to improve its stability and/or its immunogenicity; thus, it may be carried in the form of liposomes, virosomes, nanospheres, microspheres or microcapsules. The medicament or the combination of a membrane fraction of Gram-negative bacteria—biological agent can also be provided in a form which can be easily administered such as an ointment, a lotion, a solution or alternatively in the form of an adhesive composition: plaster, “patch”.

The medicament according to the invention may be used in particular for the treatment:

of microbial infections, in particular infections of the chronic type associated with the development of an ineffective specific immune response (virus: for example the human immunodeficiency virus (HIV), the hepatic viruses, in particular the A, B, C and D hepatic viruses and the parainfluenza virus, bacteria, parasites and yeasts),

of cancers, in particular in subjects carrying HIV and/or suffering from myelomas, lymphomas, leukemias, melanomas, carcinomas, of the kidney, of the brain, of the prostate, of the rectum, of the pancreas, of the ovaries, of the lung, of remission, for example.

As described above, the invention relates particularly to cellular immunotherapy using anticancer and antiinfectious vaccines in particular, by generating in vitro autologous dendritic cells, by introducing therein a tumor antigen and by reinjecting them after an optional washing step. The exposure of the dendritic cells in vitro to the membrane fraction of Gram-negative bacteria will make it possible to increase the maturation of the dendritic cells and therefore to increase the efficacy of the vaccine.

Accordingly, the present invention relates particularly to the use of a membrane fraction of Gram-negative bacteria, and more particularly of the membrane fraction of Klebsiella pneumoniae, for the preparation of a medicament for increasing the immunological response against a biological agent as defined above. This medicament may be a vaccine for the treatment or prevention of infectious diseases of viral origin—such as in particular HIV, hepatic viruses and parainfluenza virus—of bacterial or fungal origin, or caused by a yeast or a parasite.

The present invention also relates to the use of a membrane fraction of Gram-negative bacteria with at least one biological agent for the manufacture of a medicament for the treatment or prevention of cancers and in particular of cancers from myelomas, lymphomas, leukemias, carcinomas of the kidney, of the brain, of the prostate, of the rectum, of the pancreas, of the ovaries, of the lung, and for the manufacture of a medicament for the treatment or prevention of skin cancers chosen from keratinomas and carcinomas.

Indeed, in skin cancers such as melanomas and keratinomas, the cancer cells are in direct contact with the Langerhans' cells situated in the epidermis, the use according to the present invention by skin application makes it possible to act directly, locally on the Langerhans' cells. Thus, the medicament according to the invention may be applied to the skin in particular by the cutaneous, subcutaneous, transdermal or intraepidermal route, or to the mucous membranes; it then acts systemically through the maturation of the dendritic cells and locally through the maturation of the Langerhans' cells.

As described above, the treatment of these cancers may also be envisaged by injecting autologous dendritic cells, and in particular autologous dendritic cells which have been modified so as to express tumor antigens. The maturation of the dendritic cells will be brought about by the membrane fraction of Gram-negative bacteria.

The subject of the invention is also a device for the maturation of dendritic cells, such as for example a kit, comprising at least the membrane fraction of Gram-negative bacteria.

This kit may be adapted for the maturation of dendritic cells in vitro and may be used for example in research laboratories. This kit may additionally contain membrane fraction of Gram-negative bacteria of immature dendritic cells and/or the means necessary for isolating immature dendritic cells, such as for example means for purifying mononuclear blood cells. It may thus comprise for example various culture media, washing solutions, culture plates, reagents, controls such as for example antibodies, and an explanatory leaflet for carrying out the method according to the invention for the maturation of dendritic cells.

A kit according to the invention may also be adapted for carrying out the abovementioned therapeutic method. This kit may additionally contain membrane fraction of Gram-negative bacteria of immature dendritic cells and/or the means necessary for isolating immature dendritic cells, such as for example means for purifying mononuclear blood cells. It may thus comprise for example various culture media, washing solutions, culture plates, reagents, controls, and an explanatory leaflet for carrying out the abovementioned therapeutic method. It may also, where appropriate, contain heterologous antigens or means for obtaining an autologous cell lysate.

The legends to the figures and the examples which follow are intended to illustrate the invention without at all limiting its scope. These examples demonstrate the action of a membrane fraction of Gram-negative bacteria on dendritic cells: the membrane fraction of Gram-negative bacteria induces the maturation of immature human dendritic cells and confers on them potent antigen-stimulating and -presenting properties.

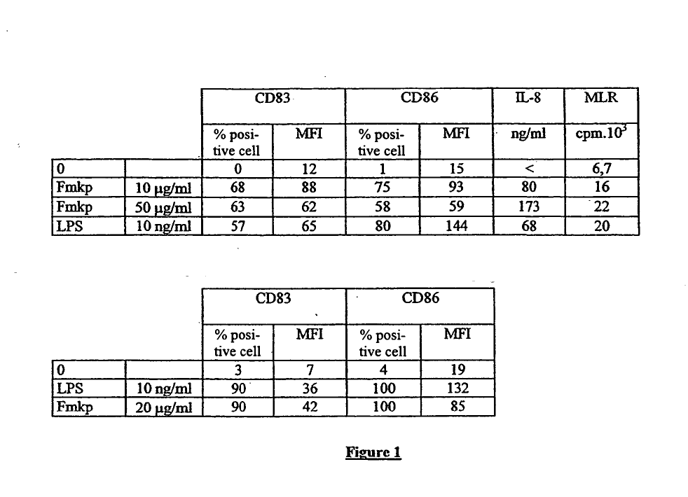

In these examples, reference will be made to the accompanying FIG. 1 which illustrates the fact that:

i) The membrane Fraction of Klebsiella Pneumonia Induces the Expression of the CD83 Molecule, Increases the Expression of CD86 and the Production of IL-8.

The results of FACS analysis of the surface molecules are expressed as MFI (mean fluorescence intensity) and are representative of one experiment out of 3. As regards CD83 and CD86, the results are also expressed as a percentage of positive cells. The results of the IL-8 assay are expressed in ng/ml and are expressed as mean ±s.d. of 4 experiments.

ii): The Membrane Fraction of Klebsiella Pneumonia Increases the T Lymphocyte Costimulating Properties (See 4th Column MLR).

Immature DCs were treated or otherwise for 24 hours with membrane fraction of Klebsiella Pneumonia or LPS. They were then irradiated and cultured with allogenic T lymphocytes of 2 different donors. The proliferation of the T lymphocytes was measured after 5 days. The results are expressed as counts per minute (cpm) and represents the mean of 5 values and are representative of one experiment out of 3.

### EXAMPLES

### Example 1

Production of a Membrane Fraction of K. pneumoniae

After thawing at +4° C. for 48 h minimum, 1 kg of dry K. pneumoniae cells are suspended at 5% dry cells. DNase is added at 5 mg/l. The mixture is then ground in a loop using a Manton Gaulin for 30 min and then clarification is carried out on SHARPLES at 50 l/h, followed by precipitation with acetic acid at pH=4.2+0.1 for 30 min. The pellet is removed (SHARPLES at 25 l/h) and the supernatant is neutralized, diluted to twice the initial volume with osmosed water. Dialysis at constant volume is then carried out on PUF 100 to 800 Ωcm, followed by concentration of the membrane suspension (MS) thus obtained to 11 l/kg of dry cells. The MS is then autoclaved at +121° C. for 35 min and it can be stored at +4° C. for 6 weeks.

Characteristics of the Membrane Fraction Obtained:

By definition, the proteoglycan titer is equal to the sum of the contents of galactose and of proteins.

Galactose: on average 2.2 g/l

Proteins: on average 10.5 g/l

### Example 2

The Membrane Fraction of Klebsiella Pneumonia Increases the Expression of CD83

A. Methodology

A.1. Generation in Vitro of Immature Human DCs.

The human dendritic cells are generated from monocytes isolated from peripheral blood. The blood is collected by leukopheresis in the presence of anticoagulant such as for example lithium heparinate. The mononuclear cells (MNC) are isolated from healthy subjects by centrifugation on a Ficoll-Hypaque gradient (density=1.077) (Amersham Pharmacia Biotech, Uppsala, Sweden).

The blood cells are centrifuged at 1500 rpm for 30 minutes at room temperature. The MNCs, located at the Ficoll-plasma interface, are recovered and washed twice in the presence of RPMI 1640 medium (Life technologies, Cergy Pontoise, France). The monocytes are purified by positive selection using a magnetic cell separator (MACS™; Miltenyi Biotex, Bergisch Gladbach, Germany) in accordance with the manufacturer's instructions. The MNCs are incubated for 20 minutes at 4° C. with magnetic beads to which anti-human CD14 mononuclear antibodies are attached. After washing, the cell suspension plus beads is deposited on a column and subjected to a magnetic field. After three washes, the column is no longer subjected to the magnetic field and the monocytes are collected by gravitation. The purity of the monocytes is evaluated by cytofluorometry (FACScan cytofluorometry; Becton Dickinson, Erembodegem, Belgium) on the basis of the parameters size-granulosity of the cells. The purity is greater than 98%. The monocytes are then cultured at the concentration of 5×10⁶ cells/ml in the following medium (called later complete culture medium): RPMI 1640 medium supplemented with 10% fetal calf serum (heating at 56° C. for 30 minutes), 2 mM L-glutamine, 50 U/ml of penicillin and 50 μg/ml of streptomycin (Life technologies) in 6-well culture plates (Nunc, Roskilde, Denmark) at the rate of 5 ml of medium per well. The cells are activated with 20 ng/ml of recombinant human IL-4 and 20 ng/ml of recombinant human GM-CSF (R&D Systems, Abingdon, United Kingdom). After 5 to 7 days of culture (37° C., 5% CO₂ in a humid atmosphere), the phenotype of the cells is defined by cytofluorometry. Briefly, one aliquot of the cell suspension is collected. The cells are washed in FACS buffer (10 mM phosphate buffer pH 7.4 containing 1% bovine serum albumin and 0.01% of sodium azide) and then distributed into wells of a 96-well culture plate with a conical bottom (Nunc) at the rate of 2×10⁵ cells in a volume of 50 ml of FACS buffer. To each well, there is added either an anti-human CD1a antibody labeled with fluorescein (Becton Dickinson) or a nonlabeled anti-human CD83 antibody revealed with an anti-mouse immunoglobulin antibody labeled with fluorescein (Becton Dickinson). After 20 minutes of incubation at 4° C., the cells are washed three times with 200 μl of FACS buffer and are then resuspended in 200 μl of this same buffer. Analysis of the expression of CD1a versus CD83 is evaluated by FACS. Only the immature dendritic cells characterized by an expression of the CD1a molecules (mean fluorescence intensity (MFI)>100) and the absence of expression of the CD83 molecule were used.

A.2. Analysis by Cytofluorometry of the Expression of the Markers of Differentiation Which is Induced by the Membrane Fraction of Klebsiella pneumoniae on Human Dendritic Cells.

The immature dendritic cells are collected, washed and then recultured in complete medium at the concentration of 10⁵ cells in a volume of 200 μl in flat-bottomed 96-well culture plates (Costar, Cabridge, USA). The cells are activated with membrane fraction of Klebsiella pneumoniae at concentrations of 10 micrograms, 20 micrograms and 50 micrograms per milliliter of final medium. 24 hours after stimulation, the expression of the CD83 and CD86 molecules is evaluated by cytofluorometry using specific monoclonal antibodies labeled with fluorescein (Becton Dickinson). The control isotypic antibodies used are obtained from Becton Dickinson. The cells are washed in FACS buffer and then distributed into wells of a 96-well culture plate with a conical bottom at the rate of 2×10⁵ cells in a volume of 50 μl of FACS buffer. An antibody is added to each well. After 20 minutes of incubation at 4° C., the cells -are washed three times with 200 μl of FACS buffer and are then resuspended in 200 μl of this same buffer. Analysis of the expression of the surface markers is evaluated by FACS.

B. Results

The results presented in FIG. 1 show that the membrane fraction of Klebsiella pneumoniae increases the expression, by the immature dendritic cells, of surface molecules involved in the activation of T lymphocytes:

As was previously demonstrated, the majority of the population of immature dendritic cells does not express CD83. The membrane fraction of Klebsiella pneumoniae induces the expression of the costimulating molecule.

The membrane fraction of Klebsiella pneumoniae also increases the expression of other molecules involved in the activation of the T lympocytes, such as CD86.

### Example III

The Membrane Fraction of Klebsiella Pneumonia Induces the Expression of IL-8 by DCs

A. Methodology:

Immature human dendritic cells were generated as described in Example 1. After 5 to 7 days of culture, the cells are recultured in complete medium at the concentration of 10⁵ cells in a volume of 200 ml in flat-bottomed 96-well culture plates. The cells are activated with membrane fraction of Klebsiella pneumoniae at concentrations of 10 micrograms and 50 micrograms per milliliter of culture medium and after 24 hours of culture the culture supernatants are centrifuged at 10 000 rpm for 15 minutes at 4° C. and IL-8 is assayed using commercial assay kits of the ELISA type (R&D Systems) according to the manufacturer's instructions.

B. Results:

The results presented in FIG. 1 show that the membrane fraction of Klebsiella pneumoniae induces expression of IL-8. It can thus be concluded that the membrane fractions of Gram-negative bacteria activate immature dendritic cells.

### Example IV

The Membrane Fraction of Klebsiella pneumoniae Increases the Dendritic Cell Costimulating Properties

A. Methodology:

A.1. Mixed lymphocytic reaction

The immature human dendritic cells are generated as described in Example 1. After 5 to 7 days of culture, the cells are washed in RPMI 1640 medium and then recultured in complete medium at the concentration of 2.5×10⁵ cells/well in a 6-well culture plate (5 ml/well). The cells are not stimulated or are stimulated in the presence of the membrane fraction of Klebsiella pneumoniae at concentrations of 10 micrograms and 50 micrograms or 10 ng/ml LPS. After 24 hours of culture, the cells are collected and washed three times in RPMI 1640 medium. The cells are then irradiated at 3000 rad. The human T lymphocytes freshly isolated from peripheral blood were prepared by the rosette technique with sheep erythrocytes. Briefly, the mononuclear cells are isolated on a Ficoll-Hypaque gradient, as described in Example 1. The mononuclear cells are resuspended in complete medium at the concentration of 200×10⁶ cells/ml and mixed with 1 ml of a suspension containing 50% sheep erythrocytes (BioMérieux, Marcy l'Etoile, France). The cell suspension is incubated at 4° C. overnight. After gentle resuspension, the T cells are isolated by centrifugation on a Ficoll-Hypaque gradient (1500 rpm for 30 minutes at room temperature). The T cell/erythrocyte complexes are collected at the bottom of the tube. The red blood cells are lysed by two successive hypotonic shocks. The purity of the cells thus obtained is evaluated by cytofluorometry using an anti-human CD3 antibody labeled with fluorescein (Becton Dickinson). The purity is >95%. After washing, the cells are resuspended in the complete culture medium at the concentration of 2.5×10⁵ cells/ml.

The activated and irradiated dendritic cells are cultured at the concentration of 10⁴ cells/200 micro-liters in a 96-well culture plate in the presence or otherwise of 5×10⁴ allogenic lymphocytes. After 5 days of culture, the proliferation of the T cells is evaluated by measuring the incorporation of tritiated thymidine (³H-Thy) (Amersham, Amersham, United Kingdom). Briefly, 0.25 mCi of ³H-Thy are added to each culture well. The incorporation of ³H-Thy is measured by a liquid scintillation counter (Packard Instruments, Australia). The results are presented in counts per minute (cpm). The mixed lymphocyte reactions are carried out with the T lymphocytes obtained from two different healthy donors.

B. Results

The results presented in FIG. 1 show that the dendritic cells stimulated by the membrane fraction of Klebsiella pneumoniae have an increased capacity to provide signals for costimulation to the T lymphocytes and induce greater proliferation of T lymphocytes than the nonstimulated DCs.

### Example V

KpOmpA Acts in Synergy With the LPS to Induce the Maturation of Dendritic Cells

Among the membrane proteins contained in FMKp (membrane fraction) is KpOmpA whose capacity to induce the maturation of human dendritic cells has already been shown (Jeannin et al., 2000, Nat Immunol, 1(6), 502-9).

However, FMKp, containing a protein level between 1.2 g/l and 3.4 g/l and a galactose level between 7.5 and 14.9 g/l, is much more effective for inducing the maturation of dendritic cells than the protein alone, as demonstrated by Table II, where the maturation of the human dendritic cells is objectified by the production of IL-12.

Thus, FMKp induces the production of 134 pg/ml of IL-12 at 0.1 μg of proteins/ml whereas among these total proteins KpOmpA represents only a small percentage (about 10%).

On the other hand, even at a high concentration (20 μg/ml), KpOmpA alone triggers the production of only 30 pg/ml of IL-12 (values extracted from Jeannin et al., op. cit.).

Dendritic cells at 10×10⁶/ml, prepared as described above, were stimulated with increasing doses of FMKp or KpOmpA. After 24 hours, IL-12, which is indicative of maturation of the dendritic cells, was assayed in the supernatants by ELISA (R&D Systems).

TABLE II
maturation of human dendritic cells in the
presence of KpOmpA or FMKp

| | IL-12 in pg/ml |
|---|---|
| KpOmpA | |
| 4 μg/ml | 10 |
| 20 μg/ml | 30 |
| FMKp | |
| 0.01 μg/ml | 55 |
| 0.1 μg/ml | 134 |
| 1 μg/ml | 241 |
| 10 μg/ml | 196 |

Without being bound by this theory, the Applicant thinks that the efficacy of FMKp in inducing the maturation of human dendritic cells can be explained by a synergistic effect between KpOmpA and the LPS contained in FMKp.

The results presented in Table III make it possible to back up this hypothesis. Dendritic cells at 10×10⁶/ml, prepared as described above, were stimulated with increasing doses of LPS in the presence or in the absence of kpOmpA (10 μg/ml). After 24 hours, IL-8 was assayed in the supernatants by ELISA (R&D Systems). The results presented are expressed in ng/ml and are representative of one experiment out of three.

TABLE III
Maturation of human dendritic cells with
LPS, in the presence or absence of KpOmpA.

| LPS (ng/ml) | without KpOmpA | with KpOmpA |
|---|---|---|
| 0 | 5 | 37 |
| 0.08 | 6 | 1130 |
| 0.4 | 467 | 2395 |
| 2 | 2050 | 4356 |

1. The use of at least one membrane fraction of Gram-negative bacteria, preferably of Klebsiella pneumoniae, comprising at least membrane proteins associated with LPSs (lipopolysaccharides), for inducing the maturation of dendritic cells.

2. The use of at least one membrane fraction of Gram-negative bacteria, and preferably of Klebsiella pneumoniae, comprising at least membrane proteins associated with LPSs (lipopolysaccharides), for the manufacture of a medicament intended for inducing the maturation of dendritic cells.

3. The use as claimed in claim 1 or 2, characterized in that said membrane fraction has a proteoglycan titer, represented by the sum of the contents of galactose and of proteins, between 1.2 g/l and 3.4 g/l for galactose and between 7.5 g/l and 14.9 g/l for the proteins.

4. The use as claimed in one of claims 1 to 3, characterized in that the generation and the maturation of the dendritic cells are carried out in vitro.

5. The use as claimed in one of claims 2 to 4, characterized in that the generation and the maturation of the dendritic cells are carried out in vitro, the mature cells then being reinjected in vivo.

6. The use as claimed in one of claims 2 to 5 and additionally of at least one biological agent for the manufacture of a medicament.

7. The use as claimed in claim 6, characterized in that the biological agent is chosen from the antigens of a bacterium, of a virus, of a yeast, of a parasite, of a fungus, tumor antigens, and the lysates of autologous and/or heterologous tumor cells.

8. The use as claimed in one of claims 2 to 5, characterized in that the dendritic cells are transfected ex vivo with a gene encoding an antigen and/or a cytokine or a growth factor.

9. The use as claimed in one of claims 2 to 8, characterized in that the medicament additionally contains a cytokine or a growth factor, preferably alpha- or gamma- interferon, TNFα, GM-CSF, IL-2, IL-4, IL-6 and IL-18 or an HSP.

10. The use as claimed in one of claims 1 to 9, for the manufacture of a medicament for the treatment or prevention of infectious diseases of viral, bacterial or fungal origin or caused by a yeast or a parasite.

11. The use as claimed in one of claims 1 to 9, for the manufacture of a medicament for combating a virus chosen from HIV, the hepatic viruses and the parainfluenza virus.

12. The use as claimed in one of claims 1 to 9, for the manufacture of a medicament for the treatment or prevention of cancers.

13. The use as claimed in either of claims 8 or 9, for the manufacture of a medicament for the treatment or prevention of a cancer from myelomas, lymphomas, leukemias, carcinomas of the kidney, of the brain, of the prostate, of the rectum, of the pancreas, of the ovaries, or of the lung, keratinomas and carcinomas.
